(19) United States
(12) Patent Application Publication
(10) Pub. No.: US 2009/0018448 A1
(43) Pub. Date: Jan. 15, 2009

(54) THREE-DIMENSIONAL ULTRASONIC DIAGNOSTIC APPARATUS

(75) Inventors: Yasutsugu Seo, Tokyo (JP); Yasuo Miyajima, Utsunomiya-Shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 12/171,518

(22) Filed: Jul. 11, 2008

(30) Foreign Application Priority Data
Jul. 13, 2007 (JP) P2007-184786

(51) Int. Cl. A61B 8/13

(52) U.S. Cl. 600/443

(57) ABSTRACT

A three-dimensional ultrasonic diagnostic apparatus has a cross-sectional image generating unit, a region-of-interest setting unit, a three-dimensional image generating unit and a display unit. The cross-sectional image generating unit generates a cross-sectional image corresponding to a desired cross-sectional plane by performing a two-dimensionally scan on the cross-sectional plane within a three-dimensional scannable region. The region-of-interest setting unit sets a second region of interest for a three dimensional image within a three-dimensional region based upon a first region of interest in the cross-sectional image. The three-dimensional image generating unit performs a three-dimensionally scan on the three-dimensional region including the second region of interest, sets a view point based upon the second region of interest, and generates the three dimensional image, including the second region of interest, in a line of sight from the view point. The display unit displays the three-dimensional image.

### BACKGROUND OF THE INVENTION

1. Field of the Invention

The present invention relates to a three-dimensional ultrasonic diagnostic apparatus which provides a three-dimensional image of the interior of an object, and particularly to a three-dimensional ultrasonic diagnostic apparatus which displays an image fused by a two-dimensional image and a three-dimensional image.

2. Description of the Related Art

In a case of diagnosing valve diseases in the cardiovascular system, for example, using conventional two-dimensional ultrasonic diagnostic apparatuses, in many cases, it is difficult to evaluate the degree of reverse flow and to determine a treatment strategy without involving comparison among a great number of cross-sectional images displayed in various directions.

In order to solve the aforementioned problems, let us consider a case of employing a three-dimensional ultrasonic diagnostic apparatus. In this case, if a user such as a doctor can observe an image in an appropriate line of sight, such an arrangement only requires such a single image, thereby solving the aforementioned problems. In particular, a three-dimensional ultrasonic diagnostic apparatus, which displays an image fused by a gray-scale structural image (two-dimensional image) and a color image of blood vessels (three-dimensional image), allows the positional relation in these images to be observed more precisely (see Japanese Patent Application Publication No. 11-164833, for example).

However, a three-dimensional scanning requires acquisition of data in volume, which is time-consuming. In some cases, this leads to a problem in that a real-time image cannot be acquired. Conversely, let us consider an arrangement directed to reproduction of an image of internal tissue with natural motion in a real-time manner. With such an arrangement, there is a need to reduce the amount of time required for scanning the overall three-dimensional region so as to improve the time resolution (volume rate). However, this involves reduction of spatial resolution (ultrasonic scanning line density), leading to either poor image quality or a limited view angle. In the latter case, the operator often cannot identify the scanned portion in the object.

In order to solve such a problem, a three-dimensional ultrasonic diagnostic apparatus has been proposed which scans only two desired cross-sectional images in the three-dimensional scannable region, instead of scanning throughout the overall three-dimensional region, thereby drastically reducing the time required for scanning (see Japanese Patent Application Publication No. 2000-135217, for example). Also, an arrangement may be made in which a composite image is generated using these cross-sectional planes and a three-dimensional image acquired in a local region set by a C-mode image. With such an arrangement, the local region is set to an optimum and smallest size, thereby improving the time resolution.

However, even if such a three-dimensional ultrasonic diagnostic apparatus is employed, in some cases, it is difficult to evaluate the degree of reverse flow and to determine treatment strategy without involving the comparison based upon a three-dimensional image displayed in various directions.

### SUMMARY OF THE INVENTION

The present invention has taken into consideration the above-described problems, and it is an object of the present invention to provide a three-dimensional ultrasonic diagnostic apparatus of the present invention which the user can comprehend a valve structure of the heart and so on in a short time.

To solve the above-described problems, the present invention provides the three-dimensional ultrasonic diagnostic apparatus, which is capable of performing a three-dimensionally scan on a three-dimensional region within an object using ultrasonic waves, comprising: a cross-sectional image generating unit configured to generate a cross-sectional image corresponding to a desired cross-sectional plane by performing a two-dimensionally scan on the cross-sectional plane within the three-dimensional scannable region; a region-of-interest setting unit configured to set a second region of interest for a three dimensional image within a three-dimensional region based upon a first region of interest in the cross-sectional image generated by the cross-sectional image generating unit; a three-dimensional image generating unit configured to perform a three-dimensionally scan on the three-dimensional region including the second region of interest, to set a view point based upon the second region of interest set by the region-of-interest setting unit, and to generate the three dimensional image, including the second region of interest, in a line of sight from the view point; and a display unit configured to display the three-dimensional image generated by the three-dimensional image generating unit.

To solve the above-described problems, the present invention provides the three-dimensional ultrasonic diagnostic apparatus, which is capable of performing a three-dimensionally scan on a three-dimensional region within an object using ultrasonic waves, comprising: a cross-sectional image generating unit configured to generate a plurality of cross-sectional images corresponding to a plurality of desired cross-sectional planes by performing a two-dimensionally scan on the cross-sectional planes within the three-dimensional scannable region; a region-of-interest setting unit configured to set a second region of interest, including a line of the intersection of the cross-sectional planes used by the cross-sectional image generating unit, for a three dimensional image within a three-dimensional region based upon a first region of interest; a three-dimensional image generating unit configured to perform a three-dimensionally scan on the three-dimensional region including the second region of interest, to set a view point based upon the second region of interest set by the region-of-interest setting unit, and to generate the three dimensional image, including the second region of interest, in a line of sight from the view point; and a display unit configured to display the three-dimensional image thus generated by the three-dimensional image generating unit.

### BRIEF DESCRIPTION OF THE DRAWINGS

In the accompanying drawings:

FIG. 1 is a flowchart which shows a processing flow of steps from a step for acquiring a tissue image and a structural image and generating a fusion image, and a step for displaying the fusion image thus generated, up to a step for handling the fusion image thus displayed, which are executed by a three-dimensional ultrasonic diagnostic apparatus according to a first embodiment of the present invention;

FIGS. 2A-2H are diagrams for describing the step for generating a cross-sectional image and the step for handling the image according to the flowchart;

FIG. 3 is a conceptual diagram shown a second region of interest including a mitral valve;

FIG. 4 is a pattern diagram shown a 3D image corresponding to the second region of interest shown in FIG. 3;

FIG. 5 is a conceptual diagram shown a second region of interest including an interventricular septum;

FIG. 6 is a pattern diagram shown a 3D image corresponding to the second region of interest shown in FIG. 5;

FIG. 7A-7H are diagrams for describing a processing flow of steps from a step for acquiring a tissue image and a structural image and generating a fusion image, and a step for displaying the fusion image thus generated, up to a step for handling the fusion image thus displayed, which are executed by a three-dimensional ultrasonic diagnostic apparatus according to a second embodiment of the present invention;

FIG. 8 is a diagram shown an example of a time sharing scanning sequence of a dislocation scanning/3D scanning according to the embodiment of the present invention; and

FIG. 9 is a diagram shown a constitution of a 3D data profile.

### DESCRIPTION OF THE PREFERRED EMBODIMENTS

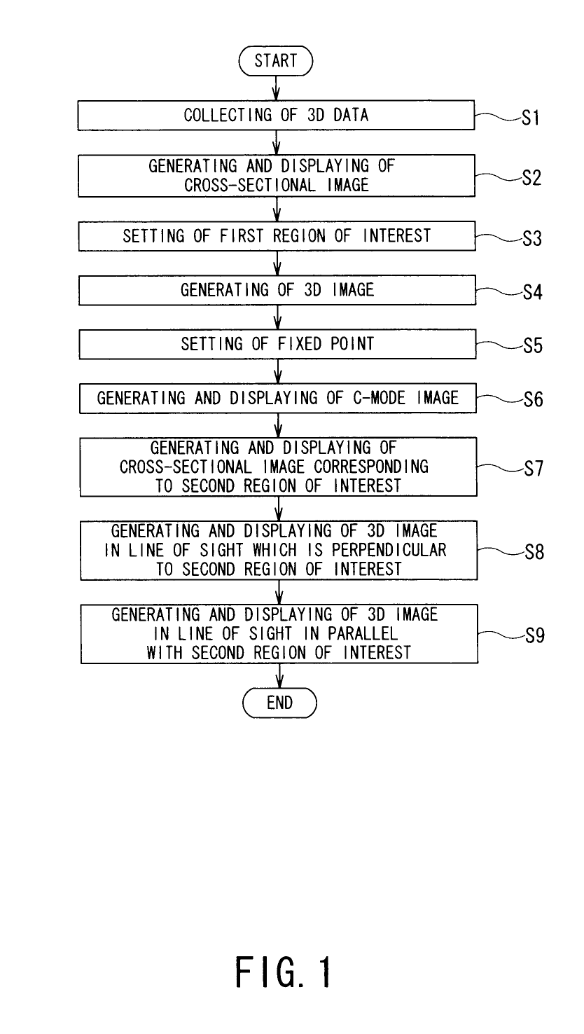

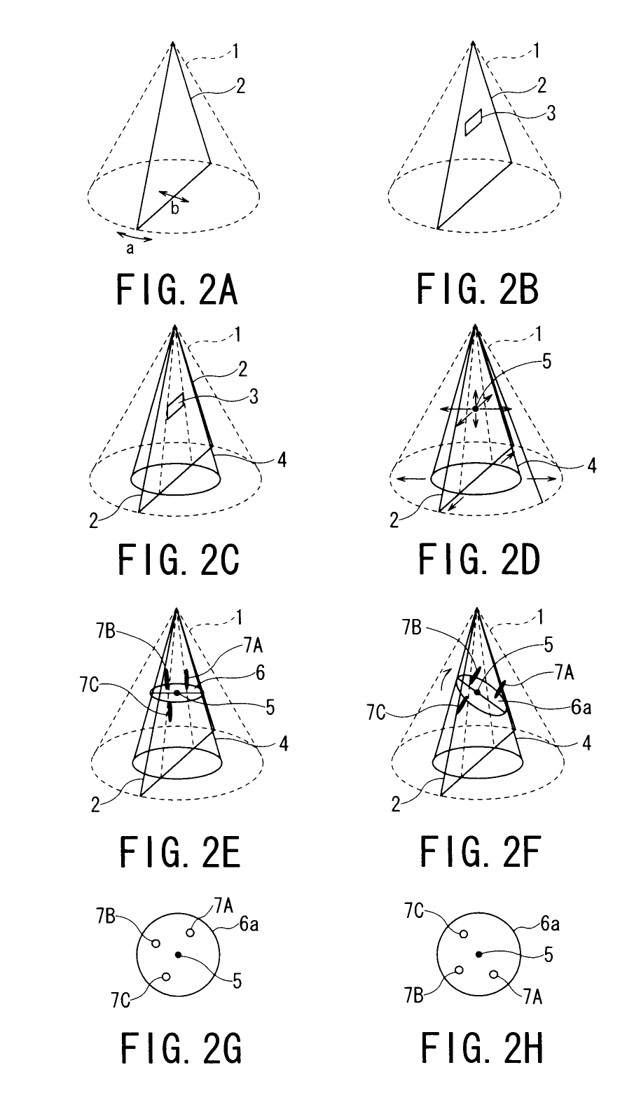

Description will be made regarding a three-dimensional ultrasonic diagnostic apparatus according to a first embodiment of the present invention with reference to the appended drawings. FIG. 1 is a flowchart which shows a processing flow of steps from a step for acquiring a tissue image and a structural image and generating a composite image, and a step for displaying the composite image thus generated, up to a step for handling the composite image thus displayed, which are executed by the three-dimensional ultrasonic diagnostic apparatus according to the first embodiment of the present invention. FIGS. 2A-2H are diagrams for describing the step for generating a cross-sectional image and the step for handling the image according to the flowchart.

First, as shown in Step S1, 3D data is acquired by the three-dimensional ultrasonic diagnostic apparatus. The data acquisition is performed according to two types of image acquisition modes, i.e., a B mode and a color Doppler mode. B-mode 2D data and color-Doppler-mode 3D data are acquired according to these image acquisition modes. B-mode data is acquired in the form of 2D data. Accordingly, the B-mode data can be acquired in a short period of time as compared with 3D data.

Next, as shown in Step S2, image processing such as a multi-planar reconstruction (MPR) processing, texture mapping, etc., is performed based upon the B-mode 2D data clipped from the 3D data thus acquired, thereby generating a cross-sectional image and displaying the cross-sectional image on a screen of a display unit. The cross-sectional image is a desired cross-sectional image 2 within a three-dimensionally scannable region 1, as shown in FIG. 2A, is displayed as a moving image on the screen. Note that when a user such as a doctor turns or tilts an ultrasonic probe while watching the cross-sectional image 2 displayed on the screen during acquiring of the B-mode 2D data, a cross-sectional plane of the cross-sectional image 2 is turned (arrow a in FIG. 2A) or is tilted (arrow b in FIG. 2A). The cross-sectional image 2 is obtained like that. Note that because the user uses an input unit such as a track ball, the cross-sectional plane can be tilted. Additionally, the cross-sectional image 2 may be an image subjected to oblique processing or a front view image, as shown in FIG. 2A.

Subsequently, in Step S3, a first region of interest 3 is set on the cross-sectional image 2. When the user moves a graphic on the cross-sectional image 2 by operating the input unit such as a mouse while watching the cross-sectional image 2 displayed on the screen during acquiring of the B-mode 2D data, the first region of interest 3 is set as shown in FIG. 2B.

Next, after the setting of the first region of interest 3, a 3D image 4 is generated based upon the 3D data acquired in the color Doppler mode (Step S4). The 3D image 4 is set to contain the first region of interest 3, as shown in FIG. 2C. This reduces a load of the acquisition of 3D data.

Subsequently, a fixed point 5 that is used when a default region 6 of a second region of interest 6a is three-dimensionally turned by step S7 to mention later is set on the first region of interest 3 (Step S5). The fixed point 5 is set in a place where the user is especially interested in in the first region of interest 3. When the user moves a graphic on the first region of interest 3 by operating the input unit such as a 3D pointing-device in a vertical direction, a horizontal direction, and a depth direction (direction orthogonal to the drawing), as shown in FIG. 2D, the fixed point 5 is set. Or, the fixed point 5 is set by a center of the first region of interest 3. This involves the reconstruction of the 3D image 4, which is performed such that the fixed point 5 is set to the central portion of a cross-sectional plane of the 3D image 4.

After the setting of the fixed point 5, a plane (C-mode plane) which is approximately orthogonal to the ultrasonic beam and which includes the fixed point 5 is generated as the default region 6, as shown in FIG. 2E. Further, the C-mode image corresponding to the default region 6 is generated based upon the 3D data of the color-Doppler-mode, and displayed on the screen (Step S6).

Next, when the user three-dimensionally turns a graphic indicated the default region 6 around the fixed point 5 as a center, as shown in FIG. 2F, by operating the input unit such as the mouse while watching the C-mode image (including a cross-sectional image corresponding to a cross-sectional plane at rotary middle) displayed on the screen during acquiring of the B-mode 2D data, the second region of interest 6a for the 3D image is set. Further, a cross-sectional image corresponding to the second region of interest 6a is generated based upon the 3D data of the color-Doppler-mode, and displayed on the screen (Step S7). Note that the default region 6 is three-dimensionally turned in a range of 360 degrees around the fixed point 5 as the center. Turning of the default region 6 is executed by dragging a point on the screen using a mouse or the like.

Next, two points as view points are set. The view points are arranged at opposed positions across the second region of interest 6a after turning by step S7. And, the view points are arranged so as to have respectively distances (corresponding to an amplification of the 3D image), corresponding to a size of an object such as a heart, from the second region of interest 6a. Further, two 3D images of the color-Doppler-mode, including the second region of interest 6a, in each line of sight from each view point is generated and displayed on the screen (step S8). Such an arrangement of the view points allows the blood vessels 7A, 7B, 7C, etc., to be observed from just a top (head side) of the second region of interest 6a, as shown in FIG. 2G. Also, such an arrangement allows these blood vessels to be observed from just a bottom (tail side) of the second region of interest 6a, as shown in FIG. 2H. Thus, such an arrangement allows these blood vessels to be observed from an optimum position, thereby facilitating the user's observation.

Further, the other view point is set. The view point is arranged on a plane surface of the second region of interest 6a after turning by step S7. And, the view point is arranged so as to have a distance, corresponding to a size of the object such as the heart, from the second region of interest 6a. And, the view point is arranged so as to have a direction of the gaze corresponding to a site such as a costa. Further, a 3D image of the color-Doppler-mode, including the second region of interest 6a, in the line of sight from the view point is generated and displayed on the screen (step S9). The 3D images generated by steps S8 and S9 is a fusion image with an organization image that indicates a substance of organs as the object, and a structure image that indicates a blood flow of the object.

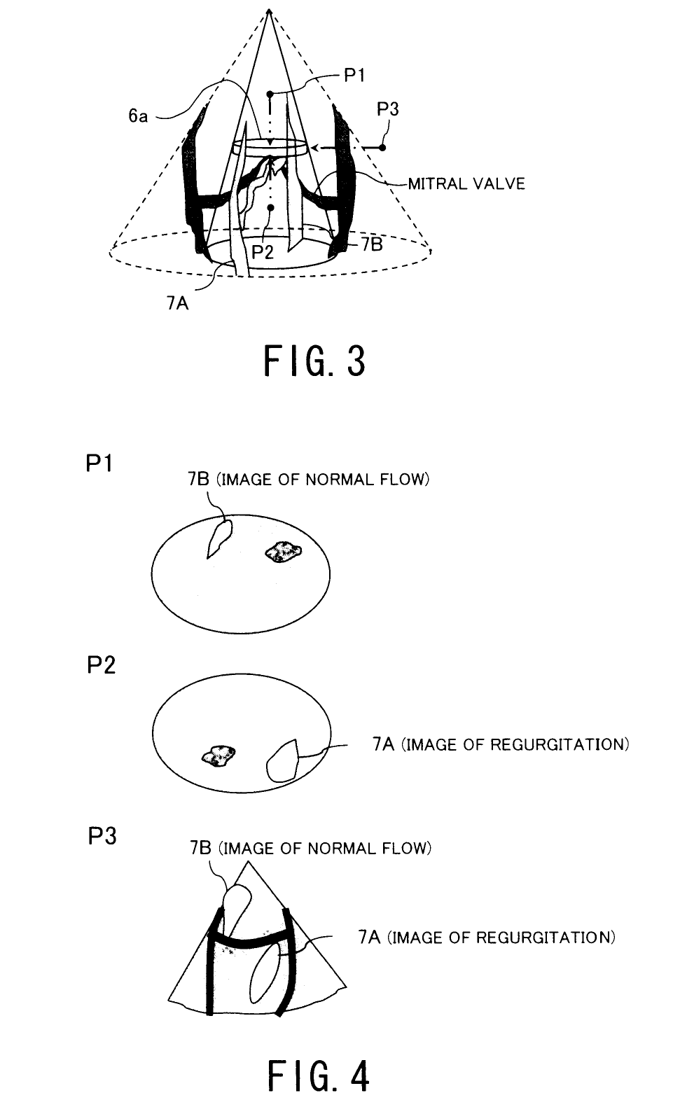

FIG. 3 is a conceptual diagram shown the second region of interest 6a, set by step S7, including a mitral valve. FIG. 4 is a pattern diagram shown the 3D image, displayed by steps S8 and S9, corresponding to the second region of interest 6a shown in FIG. 3.

The second region of interest 6a is adjusted to the mitral valve between a left ventricle of the heart (an upper side than the second region of interest 6a shown in FIG. 3) and a left atrium of the heart (an under side than the second region of interest 6a shown in FIG. 3). According to the displaying of the 3D image (shown in upper stage of FIG. 4) from the view point P1 as the head side of the second region of interest 6a among the 3D images displayed by step S8, a normal flow or a regurgitation of the blood flow is able to be displayed, it is easy for the user to be comprehended a place where the blood flow out. That is to say, the displaying of the 3D image from the view point P1 can proffer important information related to a decision of treatment policies such as a valve substitution. The blood flow in the left ventricle is able to be displayed without being covered in the organization image of the valve displayed as the fusion image, the blood flow in the left atrium is, however, is not able to be displayed.

Meanwhile, according to the displaying of the 3D image (shown in middle stage of FIG. 4) from the view point P2 as the tail side of the second region of interest 6a among the 3D images displayed by step S8, the blood flow in the atrium sinistrum is able to be displayed without being covered in the organization image of the valve displayed as the fusion image, the blood flow in the left ventricle is, however, is not able to be displayed. Whereat, it is desirable to display the 3D images from the view point P1 and the view point P2 together.

Further, according to the displaying of the 3D image (shown in under stage of FIG. 4) from the view point P3 displayed by step S9, it is easy for the user to comprehend where the blood flow arrives at from the second region of interest 6a. Because an outreach of the blood flow or a cross section based upon the 3D image from the view point P3 used for a diagnosis of the disease severity of the regurgitant jet, the displaying of the 3D image from the view point P3 is important.

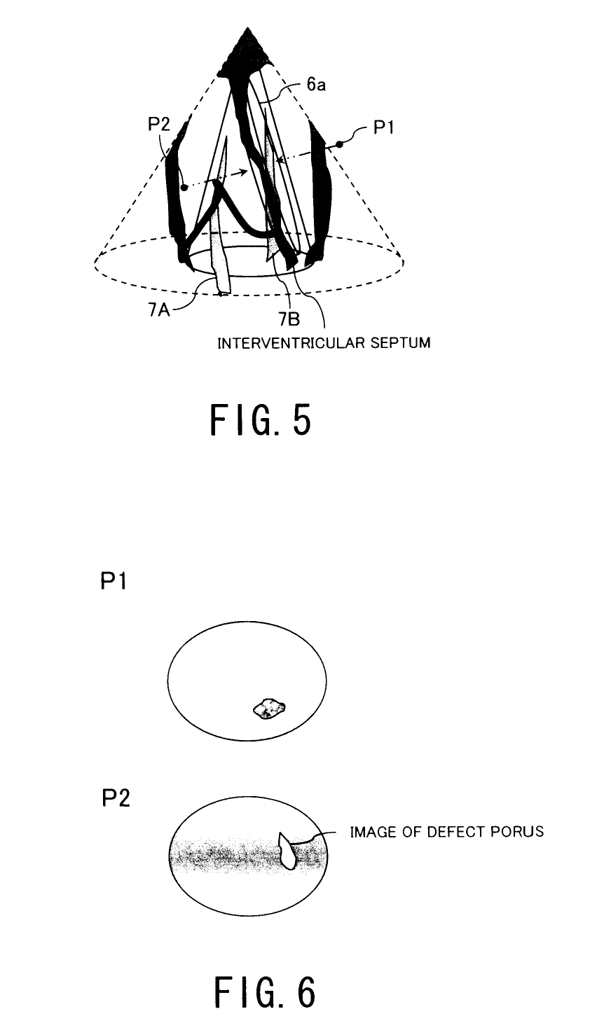

Note that FIG. 5 is a conceptual diagram shown the second region of interest 6a, set by step S7, including an interventricular septum. FIG. 6 is a pattern diagram shown the 3D image, displayed by step S8, corresponding to the second region of interest 6a shown in FIG. 5.

As for the three-dimensional ultrasonic diagnostic apparatus according to the first embodiment of the present invention, the user can comprehend a valve structure of the heart and so on in a short time, because it can be displayed immediately the 3D images, including the second region of interest 6a from 2 or 3 view points if the second region of interest 6a is set.

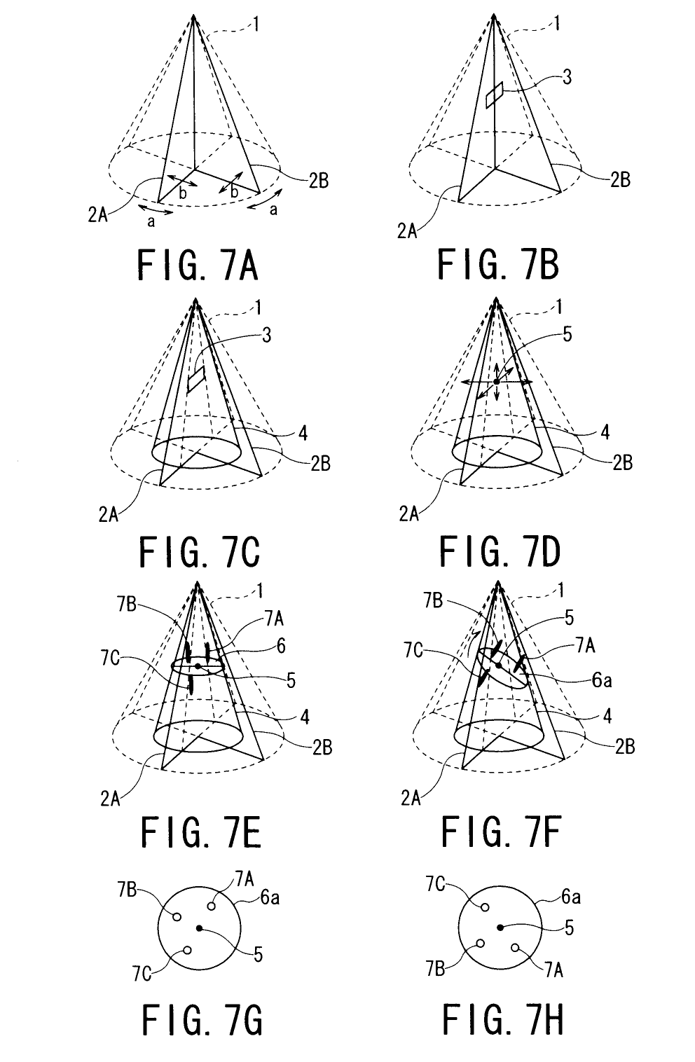

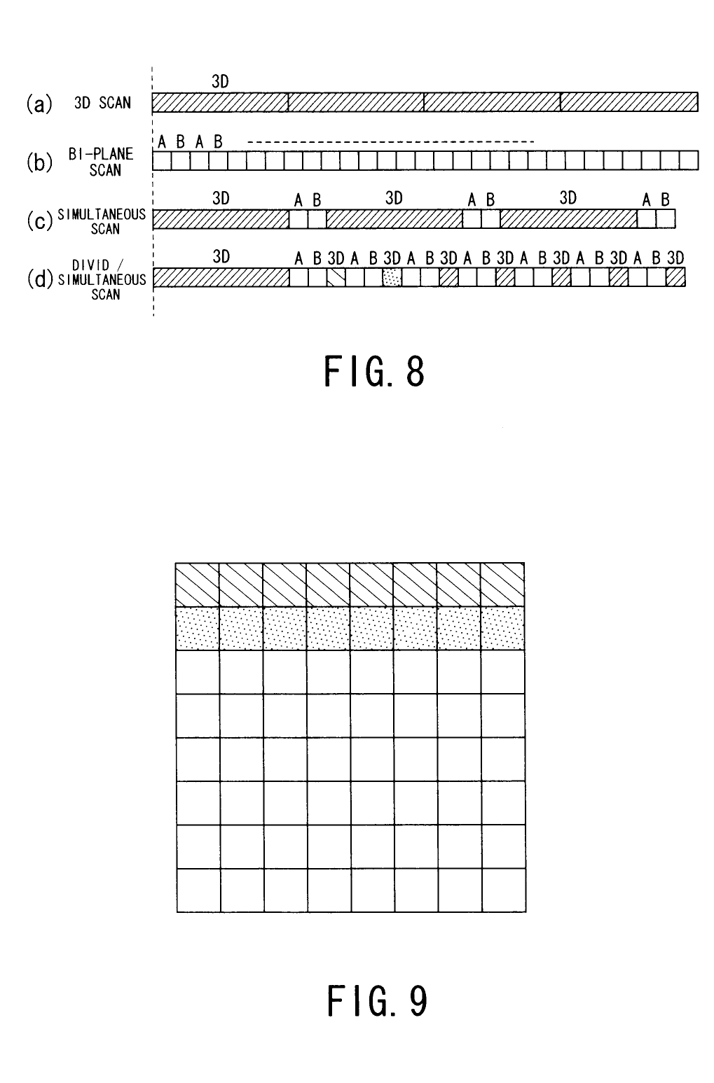

Next, description will be made regarding a three-dimensional ultrasonic diagnostic apparatus according to a second embodiment of the present invention with reference to FIGS. 7 and 8. FIG. 7 is a diagram for describing the processing from a step for generating a cross-sectional image up to a step for handling the image according to the second embodiment of the present invention.

The basic difference between the first embodiment and the second embodiment is that the three-dimensional ultrasonic diagnostic apparatus according to the present embodiment generates two cross-sectional images 2A and 2B which intersect, as shown in FIG. 7. The other components thereof are essentially the same as those of the first embodiment. Accordingly, the same components are denoted by the same reference numerals, and description thereof will be omitted.

Such an arrangement allows each of the two cross-sectional images 2A and 2B to be moved, i.e., to be turned and to be tilted. As a result, the cross-sectional images 2A and 2B intersect at 90 degrees or a desired angle. Furthermore, the first region of interest 3 is set on the line of the intersection of the two cross-sectional images 2A and 2B.

As described above, with the present embodiment, the cross-sectional images 2A and 2B are acquired by a bi-plane scan. This allows the first region of interest 3 to be set with higher precision, thereby setting the fixed point 5 with higher precision.

Furthermore, the scan ratio can be changed with respect to the scan sequence for the cross-sectional images 2A and 2B and the 3D image 4. For example, a simultaneous scan sequence, which is generally employed, may be performed, in which two-dimensional scanning for the cross-sectional images 2A and 2B and the three-dimensional scanning for the three-dimensional image 4 are alternately and repeatedly performed in a time sharing manner as shown in a third stage from the top of FIG. 8. Also, as shown in a fourth stage from the top of FIG. 8, another sequence may be employed, which performs the three-dimensional scan by dividing the 3D image.

With such an arrangement, a portion of a 3D data profile, e.g., ⅛ of a 3D data profile is replaced every three-dimensional scan, as shown in FIG. 9. This provides the advantage that the user feels that the 3D image is being updated at a faster rate.

As for the three-dimensional ultrasonic diagnostic apparatus according to the second embodiment of the present invention, the user can comprehend a valve structure of the heart and so on in a short time, because it can be displayed immediately the 3D images, including the second region of interest 6a from 2 or 3 view points if the second region of interest 6a is set.

The embodiments are described above do not intend to limit the scope of the present invention but exemplify the invention. Thus, it should be understood by a person skilled in the art that an embodiment may be made in which a part of or all the components are replaced by equivalent components, which is encompassed within the scope of the present invention.

What is claimed is:

1. A three-dimensional ultrasonic diagnostic apparatus, which is capable of performing a three-dimensionally scan on a three-dimensional region within an object using ultrasonic waves, comprising:
a cross-sectional image generating unit configured to generate a cross-sectional image corresponding to a desired cross-sectional plane by performing a two-dimensionally scan on the cross-sectional plane within the three-dimensional scannable region;
a region-of-interest setting unit configured to set a second region of interest for a three dimensional image within a three-dimensional region based upon a first region of interest in the cross-sectional image generated by the cross-sectional image generating unit;
a three-dimensional image generating unit configured to perform a three-dimensionally scan on the three-dimensional region including the second region of interest, to set a view point based upon the second region of interest set by the region-of-interest setting unit, and to generate the three dimensional image, including the second region of interest, in a line of sight from the view point; and
a display unit configured to display the three-dimensional image generated by the three-dimensional image generating unit.

2. A three-dimensional ultrasonic diagnostic apparatus according to claim 1, wherein the three-dimensional image generating unit is configured to set two view points as the view point, based upon an arrangement of the second region of interest, that are arranged at opposed positions across the second region of interest.

3. A three-dimensional ultrasonic diagnostic apparatus according to claim 1, wherein the three-dimensional image generating unit is configured to set two view points as the view point, based upon an arrangement of the second region of interest, that are arranged on a plane surface of the second region of interest.

4. A three-dimensional ultrasonic diagnostic apparatus according to claim 1, wherein the three-dimensional image generating unit is configured to generate volume data with respect to the three-dimensional region, and to generate the three-dimensional image in the line of sight by executing a volume rendering processing on the volume data.

5. A three-dimensional ultrasonic diagnostic apparatus according to claim 4, wherein the three-dimensional image generated by the three-dimensional image generating unit is a fusion image formed of a tissue image showing an organ material of the object and a structural image showing a blood flow of the object.

6. A three-dimensional ultrasonic diagnostic apparatus according to claim 1, wherein the cross-sectional image generating unit configured to generate the cross-sectional image by movably scanning at least one of the cross-sectional planes in the three-dimensional scannable region.

7. A three-dimensional ultrasonic diagnostic apparatus according to claim 1, further comprising a fixed-point setting unit configured to set a fixed point, on the first region of interest, which is used when the second region of interest is three-dimensionally turned.

8. A three-dimensional ultrasonic diagnostic apparatus according to claim 7 wherein the fixed-point setting unit is configured to set the fixed point at a center of the first region of interest.

9. A three-dimensional ultrasonic diagnostic apparatus according to claim 7, wherein the cross-sectional image generating unit configured to generate a C-mode image, including a default region of the second region of interest, approximately orthogonal to the central axis of the three-dimensional region with the fixed point set by the fixed-point setting unit as the center, and to set the fixed point by turning the default region around the fixed point as a center.

10. A three-dimensional ultrasonic diagnostic apparatus according to claim 1, wherein, in a case in which the two-dimensional scan performed by the cross-sectional image generating unit and the three-dimensional scan performed by the three-dimensional image generating unit are performed at the same time, a scan ratio can be changed.

11. A three-dimensional ultrasonic diagnostic apparatus, which is capable of performing a three-dimensionally scan on a three-dimensional region within an object using ultrasonic waves, comprising:
a cross-sectional image generating unit configured to generate a plurality of cross-sectional images corresponding to a plurality of desired cross-sectional planes by performing a two-dimensionally scan on the cross-sectional planes within the three-dimensional scannable region;
a region-of-interest setting unit configured to set a second region of interest, including a line of the intersection of the cross-sectional planes used by the cross-sectional image generating unit, for a three dimensional image within a three-dimensional region based upon a first region of interest;
a three-dimensional image generating unit configured to perform a three-dimensionally scan on the three-dimensional region including the second region of interest, to set a view point based upon the second region of interest set by the region-of-interest setting unit, and to generate the three dimensional image, including the second region of interest, in a line of sight from the view point; and
a display unit configured to display the three-dimensional image thus generated by the three-dimensional image generating unit.

12. A three-dimensional ultrasonic diagnostic apparatus according to claim 11, wherein the three-dimensional image generating unit is configured to set two view points as the view point, based upon an arrangement of the second region of interest, that are arranged at opposed positions across the second region of interest.

13. A three-dimensional ultrasonic diagnostic apparatus according to claim 11, wherein the three-dimensional image generating unit is configured to set two view points as the view point, based upon an arrangement of the second region of interest, that are arranged on a plane surface of the second region of interest.

14. A three-dimensional ultrasonic diagnostic apparatus according to claim 11, wherein the three-dimensional image generating unit is configured to generate volume data with respect to the three-dimensional region, and to generate the three-dimensional image in the line of sight by executing a volume rendering processing on the volume data.

15. A three-dimensional ultrasonic diagnostic apparatus according to claim 14, wherein the three-dimensional image generated by the three-dimensional image generating unit is a fusion image formed of a tissue image showing an organ material of the object and a structural image showing a blood flow of the object.

16. A three-dimensional ultrasonic diagnostic apparatus according to claim 11, wherein the cross-sectional image generating unit configured to generate the cross-sectional image by movably scanning at least one of the cross-sectional planes in the three-dimensional scannable region.

17. A three-dimensional ultrasonic diagnostic apparatus according to claim 11, further comprising a fixed-point setting unit configured to set a fixed point, on the first region of interest, which is used when the second region of interest is three-dimensionally turned.

18. A three-dimensional ultrasonic diagnostic apparatus according to claim 17 wherein the fixed-point setting unit is configured to set the fixed point at a center of the first region of interest.

19. A three-dimensional ultrasonic diagnostic apparatus according to claim 17, wherein the cross-sectional image generating unit configured to generate a C-mode image, including a default region of the second region of interest, approximately orthogonal to the central axis of the three-dimensional region with the fixed point set by the fixed-point setting unit as the center, and to set the fixed point by turning the default region around the fixed point as a center.

20. A three-dimensional ultrasonic diagnostic apparatus according to claim 11, wherein, in a case in which the two-dimensional scan performed by the cross-sectional image generating unit and the three-dimensional scan performed by the three-dimensional image generating unit are performed at the same time, a scan ratio can be changed.
